SEQUENCE LISTING (16 sequences)

SEQ ID NO: 1 - DNA, 20 bp, Artificial Sequence
  Description of Artificial Sequence Synthetic primer
tcaacaagcccacagggtat

SEQ ID NO: 2 - DNA, 20 bp, Artificial Sequence
  Description of Artificial Sequence Synthetic primer
actcgtgcagagcaaaggat

SEQ ID NO: 3 - DNA, 20 bp, Artificial Sequence
  Description of Artificial Sequence Synthetic primer
cgaggccatggtgctatact

SEQ ID NO: 4 - DNA, 20 bp, Artificial Sequence
  Description of Artificial Sequence Synthetic primer
acaccagggtgattcagacc

SEQ ID NO: 5 - DNA, 20 bp, Artificial Sequence
  Description of Artificial Sequence Synthetic primer
gacatggatcctgccaactt

SEQ ID NO: 6 - DNA, 20 bp, Artificial Sequence
  Description of Artificial Sequence Synthetic primer
aattccaactgccactgtcc

SEQ ID NO: 7 - DNA, 20 bp, Artificial Sequence
  Description of Artificial Sequence Synthetic primer
tcttcaagccatcctgtgtg

SEQ ID NO: 8 - DNA, 20 bp, Artificial Sequence
  Description of Artificial Sequence Synthetic primer
gcgagtctgtgtttttgcag

SEQ ID NO: 9 - DNA, 23 bp, Artificial Sequence
  Description of Artificial Sequence Synthetic primer
cccaccaatcacctgcgtacaga

SEQ ID NO: 10 - DNA, 24 bp, Artificial Sequence
  Description of Artificial Sequence Synthetic primer
ttcttggtcggtgggtgactctga

SEQ ID NO: 11 - DNA, 24 bp, Artificial Sequence
  Description of Artificial Sequence Synthetic primer
gagatgtctggaagccagaaccat

SEQ ID NO: 12 - DNA, 23 bp, Artificial Sequence
  Description of Artificial Sequence Synthetic primer
gatctcccttggggccttgaggt

SEQ ID NO: 13 - DNA, 21 bp, Artificial Sequence
  Description of Artificial Sequence Synthetic primer
tcaacatctcatgttcctccc

SEQ ID NO: 14 - DNA, 24 bp, Artificial Sequence
  Description of Artificial Sequence Synthetic primer
ttcttcactgtgggtataggtcta

SEQ ID NO: 15 - DNA, 20 bp, Artificial Sequence
  Description of Artificial Sequence Synthetic primer
ggacttcgagcaagagatgg

SEQ ID NO: 16 - DNA, 20 bp, Artificial Sequence
  Description of Artificial Sequence Synthetic primer
agcactgtgttggcgtacag

(19) United States
(12) Patent Application Publication
(10) Pub. No.: US 2008/0269173 A1
(43) Pub. Date: Oct. 30, 2008

(54) Composition for Stimulating Growth of Dermal Papilla Cells and Promoting Hair Follicle Growth Comprising Vitamin C Derivatives

(75) Inventors: Young-Kwan Sung, Daegu (KR); Moon-Kyu Kim, Daegu (KR); Jung-Chul Kim, Daegu (KR)

Correspondence Address: FOLEY & LARDNER LLP, 777 EAST WISCONSIN AVENUE, MILWAUKEE, WI, 53202-5306, US

(73) Assignee: Trichogene Inc.

(21) Appl. No.: 11/813,649

(22) PCT Filed: Jan. 6, 2006
(86) PCT No.: PCT/KR2006/000069

§ 371 Date: Jul. 10, 2007

(30) Foreign Application Priority Data
Jan. 13, 2005 (KR) 10-2005-0003091

(51) Int. Cl. A61K 31/665; A61K 31/375; A61P 17/14

(52) U.S. Cl. 514/99; 514/474

(57) ABSTRACT

Disclosed is a composition for stimulating growth of dermal papilla cells and promoting hair follicle growth comprising vitamin C derivatives.

### TECHNICAL FIELD

The present invention relates to a composition for stimulating growth of dermal papilla cells and promoting hair follicle growth comprising vitamin C derivatives.

### BACKGROUND ART

Generally, the mammalian hair follicle is a very organized, multi-layered and dynamic organ. The hair follicle includes epithelial cells such as inner and outer root sheath cells, matrix and hair shaft derived from the epithelial tissue, as well as dermal papilla and dermal sheath cell derived from the mesenchymal tissue. Interaction between the epithelial tissue and the mesenchymal tissue is necessary for hair growth after the birth, as well as development of the hair follicle.

The hair growth after the birth has a cell cycle of anagen, catagen and telogen. It has been known that the dermal papilla is encapsulated by epithelial matrix cell during the growth period, and growth factors, secreted in the dermal papilla such as insulin-like growth factor 1 (IGF-1), keratinocyte growth factor (KGF), etc., are differentiated to proliferate the epithelial cells and produce the hair shafts (Itami S, et. al., Biochem Biophys Res Commun. 212:988-94, 1995; Werner S, et. al., Science. 266:819-22, 1994).

Meanwhile, it was found that the epithelial cells undergoes programmed cell death (apoptosis) by the growth-inhibiting factors secreted in the dermal papilla such as tumor growth factor β (TGF-β) during the catagen (Soma et al., J Invest Dermatol. 111:948-54, 1998; Hibino and Nishiyama, J Dermatol Sci. 35:9-18, 2004). Accordingly, it has been known that the factors secreted in the dermal papilla cells control hair growth. Also, the size of the dermal papilla is in proportion to that of the hair follicle (Stenn and Paus, Physiol Rev. 81:449-494, 2001). Accordingly, it is expected that natural extracts or chemicals, which can control growth of the dermal papilla cells or their gene expression, play an important role in controlling the hair growth.

Vitamin C (L-ascorbic acid) exhibits a cofactor activity in the collagen synthesis, and is used as a supplementary component in the media. However, such a use of vitamin C is limited since the vitamin C is rapidly oxidized and degraded in an aqueous solution, especially under the general culture conditions. The vitamin C derivative L-ascorbic acid-2-phosphate magnesium (so-called, referred to as Asc 2-P) is a formulation that is more stable than the vitamin C, and has been used as a supplementary component in the various media. Like the vitamin C, Asc 2-P also stimulates growth of human dermal fibroblast and osteoblast (Hata et al., Eur J Biochem. 173:261-267, 1988). The vitamin C or the Asc 2-P has not been known at all to have what effect on the growth of hair and dermal papilla cells.

### DISCLOSURE OF INVENTION

Accordingly, the present invention is designed to solve the problems of the prior art, and therefore it is an object of the present invention to provide an agent for treating alopecia capable of stimulating growth of dermal papilla cells and/or hair follicles.

In order to accomplish the above object, the present invention provides a composition for stimulating growth of dermal papilla cells and promoting hair follicle growth comprising vitamin C derivatives as an effective component.

In the present invention, the vitamin C derivatives are preferably selected from the group consisting of L-ascorbic acid-2-phosphate magnesium, L-ascorbyl palmitate and L-ascorbyl stearate, and L-ascorbic acid-2-phosphate magnesium of the following Chemistry FIG. 1 is the most preferred:

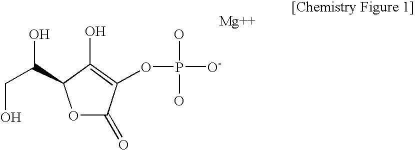

Also, the vitamin C derivatives of the present invention is characterized in that they promote growth of the hair follicles; stimulate growth of dermal papilla cells; stimulate production of an insulin like growth factor 1 (IGF-1) in the dermal papilla cells; increase expression of versican in the dermal papilla cells and the hair follicles; and increase expression of alkaline phosphatase in the dermal papilla cells.

### BRIEF DESCRIPTION OF THE DRAWINGS

These and other features, aspects, and advantages of preferred embodiments of the present invention will be more fully described in the following detailed description, taken accompanying drawings. In the drawings:

FIG. 1 is a diagram showing an effect of Asc 2-P on growth of hair follicle incubated in vitro. Length of the grown hair follicle was measured after the hair follicle was incubated for 9 days at the presence of Asc 2-P (*, P<0.05).

FIG. 2 is a diagram showing an effect of Asc 2-P on growth of the incubated dermal papilla cell. The cell number was counted using a MTT method after the dermal papilla cell was incubated for 5 days in a medium including Asc 2-P (*, P<0.05).

FIG. 3 is a diagram showing an effect of Asc 2-P on expressions of a growth factor (A), collagen (B), versican (C) and alkaline phosphatase (D) in the dermal papilla cell. Levels of mRNA were measured after the dermal papilla cell was treated with Asc 2-P for 5 days.

FIG. 4 is a diagram showing an effect of Asc 2-P on expressions of versican in the hair follicle. The hair follicle was immunostained after it was treated with Asc 2-P for 5 days.

### BEST MODES FOR CARRYING OUT THE INVENTION

Hereinafter, unlimiting examples of the present invention will be described in detail with reference to the accompanying drawings.

### Example 1

### Cell Culture and Growth of Human Hair Follicle

Biopsy samples were obtained from a laryngeal region of scalp of a patient with androgenic alopecia during the hair transplantation operation with his approval in the Kyungpook National University hospital. The hair follicles were isolated and incubated according to the known method (Philpott et al., J Cell Sci. 97:463-471, 1990).

The anagen hair follicles were isolated from the scalp under a stereoscopic binocular microscope. Upper one third of the isolated anagen hair follicles were cut out, and lower two third of them were incubated at 37° C. in a Williams E medium (Sigma, USA) under a 5% CO₂ atmosphere. The hair follicles were incubated for 9 days in a medium including Asc 2-P, and then their length was measured using a stereoscopic microscope.

### Example 2

### Cell Culture and Growth of Dermal Papilla Cell

The dermal papilla was isolated from a bulb of the severed hair follicles under the binocular microscope, and then transferred into a tissue culture dish. The dermal papilla was cultured at 37° C. for 5 days under a 5% CO₂ atmosphere in the DMEM medium supplemented with penicillin (100 U/ml), streptomycin (100 μg/ml) and 20% heat-inactivated fetal bovine serum (FBS, Hyclone, USA), and then subcultured in the same condition. The dermal papilla cells were kept in the DMEM including 10% FBS after the subculture. The secondary subcultured cells were used in the present invention.

The dermal papilla cells were incubated overnight at a density of 5,000 cells/well in a 96-well plate containing a DMEM medium supplemented with 10% FBS. The medium was then exchanged to the DMEMs including varied concentrations of Asc 2-P and incubated for 5 days, and then the cell number was measured using a MTT method.

### Example 3

### Various Gene Expressions in Dermal Papilla Cell

The dermal papilla cells were incubated for 5 days in a medium including 0.25 mM Asc 2-P, and then their RNA was isolated using a TRIzol reagent (Gibco-BRL, Grand Island, N.Y.). cDNA was synthesized from the RNA using a cDNA synthesis kit containing superscript II reverse transcriptase and random hexamer (Gibco-BRL). The cDNA was amplified with sets of gene-specific primers (Table 1). RT-PCR primers and their conditions are listed in the following Table 1.

Gene expression levels of IGF-1 (insulin like growth factor 1), HGF (hepatocyte growth factor), VEGF (vascular endothelial growth factor), KGF (keratinocyte growth factor), collagen I, collagen III, versican and alkaline phosphatase (ALP) were analyzed in the present invention.

TABLE 1

| | | | Expected | cycle | | | |
|---|---|---|---|---|---|---|---|
| Gene | Forward (5′-3′) | Reverse (5′-3′) | size | number | D | A | P |
| IGF-1 | TCAACAAGCCCACAGGGTAT | ACTCGTGCAGAGCAAAGGAT | 307 | 40 | 94 | 58 | 72 |
| IIGF | CGAGGCCATGGTGCTATACT | ACACCAGGGTGATTCAGACC | 296 | 40 | 94 | 58 | 72 |
| KGF | GACATGGATCCTGCCAACTT | AATTCCAACTGCCACTGTCC | 304 | 30 | 94 | 58 | 72 |
| VEGF | TCTTCAAGCCATCCTGTGTG | GCGAGTCTGTGTTTTTGCAG | 297 | 40 | 94 | 58 | 72 |
| Collagen I | CCCACCAATCACCTGCGTACAGA | TTCTTGGTCGGTGGGTGACTCTGA | 214 | 30 | 94 | 62 | 68 |
| Collagen III | GAGATGTCTGGAAGCCAGAACCAT | GATCTCCCTTGGGGCCTTGAGGT | 207 | 25 | 94 | 62 | 68 |
| Versican | TCAACATCTCATGTTCCTCCC | TTCTTCACTGTGGGTATAGGTCTA | 405 | 30 | 94 | 55 | 72 |
| Actin | GGACTTCGAGCAAGAGATGG | AGCACTGTGTTGGCGTACAG | 234 | 25 | 94 | 58 | 72 |

### Example 4

### Immunostaining of Versican in Hair Follicle

The hair follicles were incubated for 5 days in the media treated with 0.1 mM and 1 mM Asc 2-P, and then their immunostaining experiments were conducted against versican.

### Example 5

### Statistical Analysis

Statistical difference was analyzed using a t-test. A possibility (P) value of 0.05 or less is considered to be statistically significant in the experiments.

The inventors obtained the results from the examples as described above, as follows.

Effect of Asc 2-P on In Vitro Growth Stimulation of Hair Follicle

The inventors examined an effect of Asc 2-P on the human anagen hair follicle isolated from the scalp. It was shown that the hair follicle were significantly grown when the hair follicles were incubated for 9 days in the Williams E media including 0.05 mM and 0.25 mM Asc 2-P (FIG. 1).

Effect of Asc 2-P on In Vitro Growth Stimulation of Dermal Papilla Cell

Asc 2-P significantly stimulated growth of the dermal papilla cell when it was used at concentrations of 0.05 mM, 0.25 mM, 1 mM and 5 mM (FIG. 2). Especially, the Asc 2-P exhibited the 2.4 times higher growth of the dermal papilla cells than the control group when it was used at the concentration of 0.25 mM.

Effect of Asc 2-P on Gene Expression of Dermal Papilla Cell

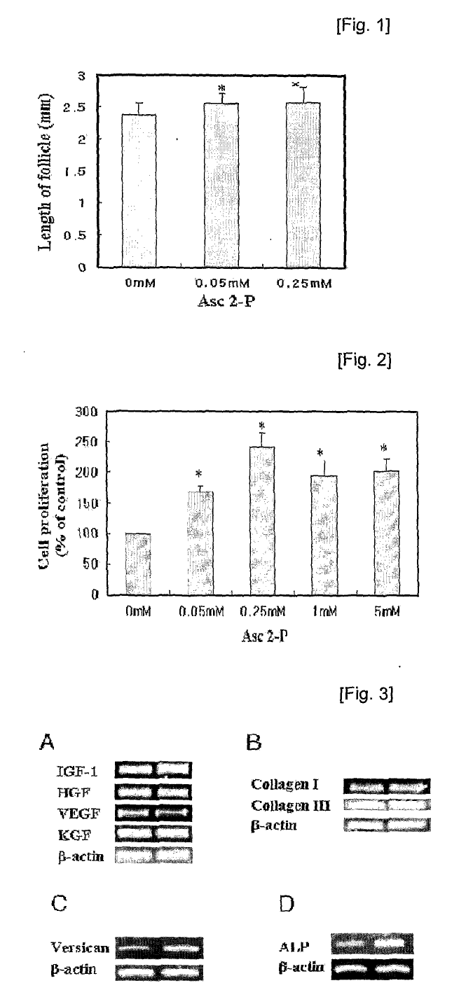

The Asc 2-P increased the concentration of mRNAs of IGF-1, versican, alkaline phosphatase (ALP) when it was used at the concentration of 0.25 mM. But, the Asc 2-P exhibited no effect on HGF, VEGF, KGF, collagen I and collagen III (FIG. 3).

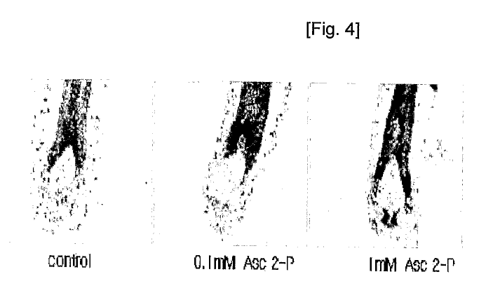

From the immunostaining experiments, it was revealed that the expression of versican was increased in the hair follicle treated with Asc 2-P (FIG. 4).

### INDUSTRIAL APPLICABILITY

As seen in the description above, the vitamin C derivative Asc 2-P of the present invention may be used as the agent for treating the alopecia since it stimulates growth of the dermal papilla cells and/or the hair follicles.

1. A composition for treating alopecia comprising vitamin C derivatives as an effective component.

2. The composition according to claim 1, wherein the vitamin C derivatives are selected from the group consisting of L-ascorbic acid-2-phosphate magnesium, L-ascorbyl palmitate and L-ascorbyl stearate.

3. The composition according to claim 1, wherein the vitamin C derivatives promote hair follicle growth.

4. The composition according to claim 1, wherein the vitamin C derivatives stimulate growth of dermal papilla cells.

5. The composition according to claim 1, wherein the vitamin C derivatives stimulate production of an insulin-like growth factor-1 in dermal papilla cells.

6. The composition according to claim 1, wherein the vitamin C derivatives increase expression of versican in dermal papilla cells and hair follicles.

7. The composition according to claim 1, wherein the vitamin C derivatives increase expression of alkaline phosphatase in dermal papilla cells.

8. The composition according to claim 2, wherein the vitamin C derivatives promote hair follicle growth.

9. The composition according to claim 2, wherein the vitamin C derivatives stimulate growth of dermal papilla cells.

10. The composition according to claim 2, wherein the vitamin C derivatives stimulate production of an insulin-like growth factor-1 in dermal papilla cells.

11. The composition according to claim 2, wherein the vitamin C derivatives increase expression of versican in dermal papilla cells.

12. The composition according to claim 2, wherein the vitamin C derivatives increase expression of alkaline phosphatase in dermal papilla cells.

13. A composition for treating alopecia comprising an effective amount of a vitamin C derivative selected from the group consisting of L-ascorbic acid-2-phosphate magnesium, L-ascorbyl palmitate and L-ascorbyl stearate and mixtures thereof for promoting hair follicle growth.

14. A composition for treating alopecia comprising an effective amount of a vitamin C derivative selected from the group consisting of L-ascorbic acid-2-phosphate magnesium, L-ascorbyl palmitate and L-ascorbyl stearate and mixtures thereof for stimulating growth of dermal papilla cells.

15. The composition according to claim 14, wherein the vitamin C derivative is L-ascorbic acid-2-phosphate magnesium.

16. The composition according to claim 14, wherein the vitamin C derivative is L-ascorbyl palmitate.

17. The composition according to claim 14, wherein the vitamin C derivative is L-ascorbyl stearate.

18. The composition according to claim 14, wherein the vitamin C derivative increases expression of versican in the dermal papilla cells.

19. The composition according to claim 14, wherein the vitamin C derivative stimulates production of an insulin-like growth factor-1 in dermal papilla cells.

20. The composition according to claim 14, wherein the vitamin C derivative increases expression of alkaline phosphatase in dermal papilla cells.
